(19) United States
(12) Patent Application Publication
(10) Pub. No.: US 2021/0077422 A1
(43) Pub. Date: Mar. 18, 2021

(54) CANNABINOID FORMULATION INCLUDING SYNERGISTIC ORGANOSULPHUR COMPOUNDS

(71) Applicant: Manu Sai Malasquez, Aurora, CO (US)

(72) Inventor: Manu Sai Malasquez, Aurora, CO (US)

(73) Assignee: Spirit Songs Botanicals, Aurora, CO (US)

(21) Appl. No.: 17/034,747

(22) Filed: Sep. 28, 2020

Related U.S. Application Data
(63) Continuation-in-part of application No. 16/824,545, filed on Mar. 19, 2020.
(60) Provisional application No. 62/820,746, filed on Mar. 19, 2019.

(51) Int. Cl. A61K 31/05; A61K 36/185; A61K 36/47; A61K 47/44; A61K 9/00; A61J 7/00

(52) U.S. Cl. CPC A61K 31/05; A61K 36/185; A61J 7/0053; A61K 47/44; A61K 9/0056; A61K 36/47

(57) ABSTRACT

A cannabinoid composition for reducing the pathogenic microbial load of a subject. The cannabinoid composition includes purified water, full spectrum hemp extract having a between 30-70% cannabidiol concentration, which is predominately Cannabidiol, 200-800 mg and bioactive amounts of crofelemer and bioactive amounts of organosulfur compounds. Preferably, the Crofelemer is derived from Ajo Sacha paste, which naturally includes bioactive amounts of Crofelemer extracted from the latex of Croton lechleri. The paste is dissolved in the water in a final concentration of 600 mg paste per 60 ml (2 oz) finished product. The organosulfur preferably is derived from Sangre de Grado and added in a standardized aqueous commercial form at a concentration of 0.5 ml per 60 ml of the finished product and an excipient. This yields a cannabinoid composition with detectable and bioactive amounts of Crofelemer and organosulfur compounds. The cannabinoid composition is packaged in an oral dropper bottle.

The present invention in a continuation-in-part of U.S. application Ser. No. 16/824,545, filed Mar. 19, 2020 which claims benefit of Provisional Application No. 62/820,746, filed Mar. 19, 2019, the content of both applications is herein incorporated in their entirety.

### FIELD OF THE INVENTION

The present invention relates to botanical compositions including cannabinoids. More particularly, the invention relates to a cannabinoid composition including hemp extract, Ajo Sacha and Sangre de Grado for treating various diseases and disorders.

### Background and Summary of the Present Invention

The pharmacological and therapeutic properties of Ajo Sacha are undergoing substantial discovery over the last few decades and are subject to a growing amount of scientific research.

Ajo Sacha (mansoa alliacea) is wild jungle garlic said to have originated in the Amazon rainforest, but it has since spread to several other parts of the world with similar climates. Ajo Sacha contains sulfur compounds that garlic does, such as allicin and ursolic acid.

The sulfur compounds (alliin and various allyl sulfides) in both garlic and Ajo Sacha have been studied by many and reported over the years to be able to lower cholesterol. The scientists reported that when laboratory rats are fed dried Ajo Sacha flowers (approx 2% of their dietary intake), the cholesterol levels are lowered, and much like garlic, the absorption of cholesterol in the intestines was inhibited. In 2001, a research group reported that the Ajo Sacha's organosulfur compounds proven to lower the cholesterol in both humans and animals in their study.

In research published in 1980, a water extract of Ajo Sacha leaves was reported to have an antioxidant effect which was attributed to the anthocyanin compounds found in the plant. These antioxidant actions were independently reported by other researchers in studies published in 1995, 1997, 2009 and 2017. Several of these studies reported very strong antioxidant actions and then attributed them to organosulfur compounds and ursolic acid.

Researchers first confirmed Ajo Sacha's long standing use for arthritis and rheumatism when they reported that the plant was capable of inhibiting COX (an enzyme required in the inflammatory process) and well as reduced ear edema in a study with rats in 1997. Other studies were published in 2009 and 2018 confirming Ajo Sacha's anti-inflammatory actions. The newer research is reporting that Ajo Sacha's anti-inflammatory actions can also be explained by the plant's ability to modulate the immune system and reduce the production of immune cells that cause inflammation. In a study in 2018, it was additionally reported very strong pain-relieving effects in mice that should be studied further.

Ajo Sacha has also been reported with antimicrobial actions against fungi, plant viruses, and bacteria which may help explain its long-standing use for colds, flu, pneumonia and other upper respiratory infections. Various different studies from 1970 to 2012 have reported Ajo Sacha's beneficial effects against bacteria, viruses and fungi. One of these studies published in 2005 said the antifungal action of a crude Ajo Sacha leaf extract equaled that of a leading antifungal drug (clotrimazole) at very low dosages. These researchers also tested the leaf extract against head lice and lung cancer cells with very positive results for both at very low dosages. They summarized their research saying Ajo Sacha should continue to be studied for possible new antifungal, cancer and anti-lice drugs based on the results they achieved. In addition to killing lice, other research published in 2016 reported an essential oil of Ajo Sacha leaves was highly effective at both killing and repelling white flies that are a problem pest in commercial tomato crops in South America.

Further, Ajo Sacha has been studied for its anti-cancer actions in three studies in addition to the one study referenced above. Researchers first reported in 1992 that Ajo Sacha's anti-cancer actions came from the well-known and well-studied anti-cancerous naphthoquinones chemicals found in the plant. But research published in 2015 indicates that the anti-cancerous actions could also be coming from the organosulfur compounds. A third research group published a preliminary in vitro study in 2015 that just showed a crude leaf extract was active at low dosages against a mouse cancer cell line.

Hence from the several studies, it has been proven that Ajo Sacha provides Antimicrobial effects, Anti-inflammatory effects, Antioxidant effects, Cellular Protective effects etc.

Similarly, the pharmacological and therapeutic properties of Sangre de Grado are undergoing substantial discovery over the last few decades and are subject to a growing amount of scientific research.

The results of in vitro and in vivo studies largely support the majority of ethnomedical uses of Sangre de Grado including the treatment of diarrhea, wounds, tumors, stomach ulcers, herpes infection, the itching, pain and swelling of insect bites, and other conditions. Clinical studies of Sangre de Grado products have reported positive results in the treatment of traveler's and watery diarrhea and the symptoms of insect bites. Sangre de Grado (SDG) is a red viscous sap and/or latex/extract from the lechleri Croton tree.

Because the sap has shown low toxicity and preparations used in clinical studies were well tolerated, further clinical and pharmacologic studies are anticipated.

Sangre de Grado is botanically classified in the Family—Euphorbiaceae, Genus—Croton, and species—lechleri. Sangre de Grado's red sap or latex (and also its bark) has a long history of indigenous use in the rainforest and in South America. The earliest written reference dates its use to the 1600s, when Spanish naturalist and explorer P. Bernabé Cobo found that the curative power of the sap was widely known throughout the indigenous tribes of Mexico, Peru, and Ecuador. For centuries, the sap has been painted on wounds to staunch bleeding, to accelerate healing, and to seal and protect injuries from infection. The sap dries quickly and forms a barrier, much like a “second skin.” It is used externally by indigenous tribes and local people in Peru for wounds, fractures, and hemorrhoids, internally for intestinal and stomach ulcers, and as a douche for vaginal discharge. Other indigenous uses include treating intestinal fevers and inflamed or infected gums, in vaginal baths before and after childbirth, for hemorrhaging after childbirth, and for skin disorders.

Scientists have attributed many of the biologically active properties of the sap (especially its wound-healing capacity) to two main “active” constituents: an alkaloid named taspine, and a lignan named dimethylcedrusine.

Of course, botanists, herbalists, and naturopaths would disagree with such reductionist conclusions and often do in this particular case; the matter is actually proven by science. Noted author and ex-USDA economic botanist Dr. James Duke summed this up eloquently, saying, “I like the comments on dragon's blood, and would add one further note: in addition to the proanthocyanadins (including Pycnogenol) and taspine, there's another active ingredient—dimethylcedrusine. While each of these alone—dimethylcedrusine, Pycnogenol and taspine—was shown to effectively heal wounded rats (with squares of skin exfoliated, i.e., peeled off) by European scientists, the whole dragon's blood was shown to speed healing four times faster. The whole was better than the sum of its parts. Synergy makes the whole herb stronger; diversity makes the rainforest stronger.”

The wound-healing action of Sangre de Grado resin was first related to the taspine alkaloid in 1989. Several later studies also concentrated on the wound-healing and antitumorous properties of taspine. The lignan dimethylcedrusine was isolated by scientists in 1993 and was shown to play a central role in Sangre de Grado's effective wound-healing action. This Belgian study revealed that the crude resin stimulated contraction of wounds, helped in the formation of a crust/scab at the wound site, regenerated skin more rapidly, and assisted in the formation of new collagen. This was the study to which Dr. Duke referred in documenting that the crude resin was found to be four times more effective at wound healing and collagen formation than its isolated chemicals (and healed wounds 10-20 times faster than using nothing at all).

The Belgian scientists also determined that taspine was active against herpes virus in this study. In 1994 other phytochemicals were found, including phenolic compounds, proanthocyanadins, and diterpenes, which showed potent antibacterial activity (against E. coli and Bacillus subtilis) as well as wound-healing properties. Another study documented Sangre de Grado's antioxidant effects and researchers in Canada documented its antifungal properties. Another important traditional use of the sap was verified by clinical research in a 2000 study designed to evaluate its gastrointestinal effects. Researchers concluded that “Sangre de Grado is a potent, cost-effective treatment for gastrointestinal ulcers and distress via antimicrobial, anti-inflammatory, and sensory afferent-dependent actions.” In 2002, these same researchers reported that Sangre de Grado evidenced an in vitro effect against stomach cancer and colon cancer cells as well. In 2003 Italian researchers reported that the resin inhibited the growth of a human myelogenous leukemia cell line and also prevented cells from mutating in test tube studies.

In addition, several health practitioners in the U.S. indicate benefits in using Sangre de Grado resin internally for diabetic neuropathy because of its previously documented effects on nerve endings, nerve pain and nerve inflammation. Benefits have also been reported with diabetes-related skin ulcers and sores (applied topically) which have refused to heal using other methods.

Hence from the several studies, it has been proven that Sangre de Grado provides antibacterial and antiviral, analgesic effects, anti-inflammatory effects, gastrointestinal effects, immunomodulating effects, wound-healing effects etc.

Similarly, cannabinoids are known for use in therapeutic treatment of disease, disorder or various medical conditions. Cannabinoids includes analgesic, anti-inflammatory, anticancer, antibiotic, anti-anxiety, and anti-oxidant properties and used for antiepileptic effects, anti-inflammatory effects, immunomodulating effects, neuroprotective effects etc.

Cannabidiol (CBD) is a cannabinoid derived from Cannabis Sativa L. Hemp is Cannabis Sativa L. having less than 0.3% Tetrahydrocannabinol (THC) by weight, and is defined in the 2018 Farm Bill in the United States.

Therefore, the present invention aims at providing botanical blends including cannabinoids, Ajo Sacha and Sangre de Grado for treating various diseases and disorders. The present invention, when consumed orally, also reduces the pathogenic microbial load of the subject.

### PREFERRED EMBODIMENTS

Provided herein is a pharmaceutical composition i.e. cannabinoid composition. The cannabinoid composition includes purified water, hemp extract having a between 30-70% cannabidiol concentration, 200-800 mg of Ajo Sacha paste dissolved in the water in a preferred concentration of 600 mg paste per 60 ml (2 oz) finished product, Sangre de Grado is added in a standardized aqueous commercial form at a concentration of 0.1-1.0 ml per 60 ml of the finished product and an excipient chosen from group consisting of emulsifiers, preservatives, fragrance, flavorings, a carrier oil, and combinations thereof. The concentration of each of these ingredients can be varied

In one aspect of the present invention, the carrier oil selected from a group consisting of olive oil, avocado oil, coconut oil, and combinations thereof.

In one aspect of the present invention, the cannabinoid composition may include cannabidiol, cannabidiolic acid, tetrahydrocannabinol, and its acid form. Further, CBN, CBG and other common cannabinoids derived from Cannabis sativa L. The cannabinoids may be in the form of a whole plant extract having traces of many cannabinoids, or a blend of isolated terpenes, plant flavonoids, and isolated cannabinoids.

In one aspect of the present invention, the cannabinoid composition is formulated into a topical cream, an oral spray, oral tincture, or dissolvable drops for time release delivery via oral mucosal membranes.

Another aspect of the present invention provides a method of manufacturing of cannabinoid composition includes dissolving equal amounts of 30×Ajo Sacha paste in equal weight hot purified water, adding correct amounts of CBD oil, adding commercial Sangre de Grado solution and adding Ajo Sacha solution. Further, adding a small amount of carrier oil with necessary excipients, emulsifiers, fragrance, preservatives, flavors, and colors, blending mixture following standard pharmaceutical compounding procedures and packaging and shipping following standardized acceptable methods.

It will be understood that certain ingredients can be added to the compositions described herein without materially affecting the basic and novel properties of the compositions described herein. For example, the compositions can include undisclosed and/or unclaimed ingredients that do not materially affect the basic and novel properties of the compositions described herein, therapeutic or otherwise. Examples of such ingredients include flavorings and sweeteners that provide a more pleasant taste and/or odor, but do not materially affect the desired properties, therapeutic or otherwise, of the compositions described herein.

Other variations, embodiments and features of the present disclosure will become evident from the following detailed description, abstract and claims.

### DETAILED DESCRIPTION

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise.

The term “pharmaceutical composition or composition” includes the compositions described herein and any additional components that are desired for use of the composition to a user or for use of the composition by a user.

The term “treating” or “treatments” or “prevention” as used herein can include any of the following: alleviating, reducing, improving, mitigating, or eliminating a disease, disorder or medical condition.

As used herein, “therapeutically effective amount” refers to administration of an amount of a given compound, to a subject in need thereof that achieves the desired therapeutic effect.

Although Cannabidiol (CBD) is described in the present invention, Cannabidiol (CBD) could be supplemented with any of the hundreds of cannabinoids found in Cannabis Sativa L. including hemp-derived cannabinoids.

As used herein, a cannabinoid is defined as any molecule that impacts either the CB1 or CB2 receptors in humans. Cannabis Saiva L is rich in phytocannabinoids, but the term “cannabinoids” also includes synthetic cannabinoids and endocannabinoids.

Embodiments of the present invention provide botanical blends of a cannabinoid composition including cannabidiol (CBD), Ajo Sacha paste dissolved in water, Sangre de Grado and at least one excipient. In one embodiment, the cannabinoid composition includes cannabidiol (CBD), 60 ml of water, 200-800 mg of Ajo Sacha paste dissolved in the water in a final concentration of 600 mg paste per 60 ml (2 oz) finished product. Sangre de Grado is added in the standardized aqueous commercial form at a concentration of preferably 0.5 ml per 60 ml of finished product and an excipient chosen from the group consisting of emulsifiers, preservatives, fragrance, flavorings, a carrier oil, and combinations thereof.

The cannabinoid composition may include cannabidiol, cannabidiolic acid, tetrahydrocannabinol, and its acid form. CBN, CBG and other common cannabinoids derived from Cannabis sativa 1. The cannabinoids may be in the form of a whole plant extract having traces of many cannabinoids, or a blend of isolated terpenes, plant flavonoids, and isolated cannabinoids.

In one embodiment, one dropperful of the botanical blends of the cannabinoid composition contains 1 ml or 13.3 mg of cannabidiol (CBD).

Embodiments of the present invention, the cannabinoid composition can be formulated into a topical cream, an oral spray, oral tincture, or dissolvable drops for time release delivery via the oral mucosal membranes.

In the embodiments, the cannabinoid composition drops for relief of pain and inflammation.

Further, the amounts of active ingredients, carriers, inactive ingredients can individually vary by up to 20% from each amount specified herein, whether expressed on a percentage basis, or by weight, or by volume.

In some embodiments, the carrier oil can be synthetic or a combination of natural oils, having a preferred lipid profile.

All there reliable herbs (Cannabis Saiva L, Ajo Sacha and Sangre de Grado) used around the world by both indigenous and modern civilizations is now proposed to be formulated together from their complete plant derived extracts to make a synergistic medicinal supplement that is convenient and effective for the relief of pain, inflammation, wound healing, and other chronic disease states. The long and well documented history of each of the ingredients by themselves have demonstrated the viability of using these herbal treatments over hundreds of years of documented history, with the added benefit of updated pharmaceutical manufacturing processes that both enhance the concentration of active ingredients along with the added comfort of pleasing formulations to the taste and presentation for modern sensibilities.

Cannabidiol (CBD) products by themselves are now well tolerated and acceptable to patients around the world who provide ample testimony to their effectiveness and safety, but all products have room to improve.

Therefore, this novel cannabinoid composition of Cannabidiol (CBD), Sangre de Grado (Dragons Blood) and Ajo Sacha (Garlic Tree) has independent biological properties that effectively assist the treatment of many chronic conditions more powerfully than any of the constituents alone.

The cannabinoid composition of the present invention is described here with various examples having varied composition without departing from the scope of the invention.

### Example 1

In one exemplary embodiment, the cannabinoid composition is of 30 ml Ajo Sacha/CBD tincture in Spirit Song proportions including 40% CBD (400 mg per bottle), 60% Ajo Sacha (600 mg per bottle), 0.5 ml Sangre de Grado (per bottle) and carrier Oil selected from the group consisting of olive oil, avocado oil, coconut oil, and combinations thereof.

### Example 2

In another exemplary embodiment, the cannabinoid composition is of Spirit Song cannabidiol (CBD) tincture in a dropper bottle with a dropper to enable drops of the cannabinoid composition for oral delivery. The composition for each 1 ounce bottle contains high quality, organic, CBD oil (full-spectrum CBD hemp oil contains CBD plus over 200 natural compounds and nutrients including Terpenes (limonene, myrcene, pinene, humulene, linalool, beta-caryophyllene, and others) with no THC, obtained from a reputable source tested for heavy metals and pesticides to achieve a total of 400 mg CBD per bottle; Ajo Sacha, Mansoa alliacea, or garlic vine, obtained from a thick paste of desiccated tar of the whole plant to deliver 300 mg of extract per 1 oz of cannabinoid composition. The Sangre de Grado, in one embodiment, is obtained from Croton lechleri as a standardized aqueous solution to deliver 0.25 ml. extract per 1 oz of cannabinoid composition. The CBD as defined herein is the sum of cannabidiol and cannabidiolic acid and other isomers of cannabidiol.

In an alternate embodiment of Example 2, the amount of Sangre de Grado is 0.5 ml per 1 oz bottle. The amount of Ajo Sacha paste is 600 mg per 1 oz bottle. It can be appreciated that the amount of Sangre de Grado can vary between 0.25-0.5 ml per 1 oz of cannabinoid composition. Also the amount of Ajo Sacha can vary between 300-800 mg per 1 oz of cannabinoid composition.

Crofelemer is a purified proanthocyanidin oligomer extracted from the sap of Croton lechleri. It exhibits significant antibacterial and antiviral properties. Such antiviral activity includes indications with influenza, parainfluenza, herpes simplex viruses I and II, and Hepatitis A and B. Crofelemer is found in trace amounts in various embodiments of the present invention, and may be concentrated to between 0.001 and 2.3% of the formulation of the present invention.

Then all active extracts are mixed in an appropriate compounding laboratory with a Carrier Oil to allow maximum bioavailability using appropriate surfactant emulsifiers, preservatives and flavors to enhance palatability and patient acceptance.

### Example 3

In another exemplary embodiment, the cannabinoid composition is of Spirit Song™ CBD oil combination. Each batch of Spirit Song™ CBD oil combination contains a 1 ounce dropper bottle 40% CBD (400 mg per bottle), 60% Ajo Sacha (600 mg per bottle), 0.5 ml Sangre de Grado per bottle and at least one carrier oil solution. The CBD oil is preferably a whole plant extract from cannabis saliva l., having less than 0.3% tetrahydrocannabinol (Hemp), containing various trace cannabinoids in addition to CBD, which is the predominant cannabinoid.

In another embodiment, the present invention provides a method of manufacturing of Cannabidiol (CBD) Drops. The method includes (i) dissolve equal amounts of 30×Ajo Sacha paste in equal weight hot purified water, (ii) add correct amounts of CBD oil, commercial Sangre de Grado solution, and Ajo Sacha solution to the proprietary carrier oil, with necessary excipients, emulsifiers, fragrance, preservatives, flavors, and colors, (iii) blend mixture following standard pharmaceutical compounding procedures and (iv) packaging and shipping following standardized acceptable methods.

Specific manufacturing techniques, emulsifiers, preservatives, fragrance, packaging, colors, and flavors depending on choice of third party compounding laboratory chosen may slightly change if needed. Different concentrations of active ingredients may be adjusted as experience necessitates or demand requires.

The composition is administered by mouth, in the recommended dose of 2 droppers full, two times a day. Further, in some embodiment it is recommended that administering with ¼ dropper (0.25 ml) twice daily with food, preferably morning and bedtime and gradually increases to ½ dropper (0.5 ml) twice per day. Make sure to start low and go slow. Use the same dose for several days. Observe the effects and if necessary adjust the amount.

### Example 4

A topical salve packaged in a tin having a threaded lid and holding 2 ounces of salve. The salve includes 1000 mg Ajo Sacha, 1000 mg of Sangre De Grado, and 400 mg CBD in a paraben free base. The CBD oil is extracted using CO₂ extraction in a hole plant extract. The base includes a blend of oils selected from coconut, olive avocado, and primrose oil. Beeswax and Jojoba are also included. Andean mint is added for aromatic qualities and synergy with the active ingredients to improve bioavailability. This salve has particular antiseptic, anti-fungal and anti-microbial properties that accelerate healing of topical wounds, cuts, scrapes, and chronic dermatitis.

The ratios on a per ounce basis are equal amounts of Ajo Sacha and Sangre De Grado (500 mg) and 200 mg of CBD per ounce. This ratio has documented healing effects. While the ratio of these active ingredients is 5:5:2, these each can be modified by 20% in accordance with various embodiments of the invention.

### Bio-Efficacy

Cannabidiol (CBD) oil is a major active ingredient with a litany of proven medical effects including natural pain relief and anti-inflammatory properties, quitting smoking and drug withdrawals, epilepsy, neurological symptoms and disorders, fighting cancer, anxiety disorders, acne, Alzheimer's disease, all documented by extensive scientific publishing.

The present invention includes detectable amounts of Crofelemer. Preferably, the Crofelemer comprises between 0.001% and 2.3% of the cannabinoid composition on a weight to weight basis. Crofelemer is an active component of Sangre de Grado and is a complex mixture of procyanidins and prodelphinidins having a molecular mass of up to 9 kDa. It is known to treat microbial infections of the digestive tract, and many other conditions. The present invention is intended to reduce the viral and bacterial load of a subject, and thus free the immune system to function more effectively. Thus the cooperation of all active ingredients, and its complex chemical constituents synergistically achieve this goal of reducing the overall pathogenic microbial load of the subject. The CBD oil, and its components in addition to cooperating with the other active ingredients as an antimicrobial component, provides an analgesic effect to ease pain in a subject.

Ajo Sacha, Mansoa alliacea, or garlic vine are another well documented herbal treatment for a variety of conditions, including lower cholesterol, very strong antioxidant actions, arthritis and rheumatism, antimicrobial actions against fungi, plant viruses, and bacteria which may help explain its long-standing use for colds, flu, pneumonia and other upper respiratory infection, anti-cancer actions came from the well-known and well-studied anti-cancerous naphthoquinones.

Ajo Sacha's anticancer actions came from the well-known and well-studied anti-cancerous naphthoquinones chemicals found in the plant. Further, it is found that the anti-cancerous actions could also be coming from the organosulfur compounds. Naphthoquinones are found in the Ajo Sacha, also other names are available as 1,4-Naphthoquinone, 1-Naphthol.

Further, Ajo Sacha's plant contains high amounts of organosulfur, flavonoids, and tannins which all have been shown to exhibit antioxidant, anti-inflammatory, and antibacterial actions. It is these biological actions that set the intention of including Ajo Sacha in the composition of the present invention.

Further, the organosulfur compounds found in Ajo Sacha (Mansoa alliacea) plant derivatives include diallyl disulphide, diallyl trisulphide, alliin, allicin, propylallyl, divinyl sulfide, diallyl sulfide, dimethyl sulfide, daucosterol, beta-sitosterol, fucosterol, stigmasterol, iridoides and isothiocyanates, naphthoquinones, alkaloids, saponins, flavones etc. In alternate embodiments of the invention Ajo Sacha can be replaced with an engineered balance of the aforementioned organosulfur compounds as isolated ingredients to yield detectably improved efficacy. In a further variation of the invention, specific isolated organosulfur components or ursolic acid, or combinations thereof can be included as additives to achieve maximum efficacy of the present invention.

As known the benefit of curative power of the Sangre de Grado's red sap, the sap dries quickly and forms a barrier, much like a “second skin”, internally for intestinal and stomach ulcers, taspine was active against herpes virus in this study. In 1994 other phytochemicals were found, including phenolic compounds, proanthocyanadins, and diterpenes, which showed potent antibacterial activity (against E. coli and Bacillus subtilis), several health practitioners in the U.S. indicate benefits in using Sangre de Grado resin internally for diabetic neuropathy because of its previously documented effects on nerve endings, nerve pain and nerve inflammation.

Hence, alternative to utilizing raw sap from Sangre de Grado, the sap constituents including stapine, phenolic compounds, diterpenes, proanthocyanadines, and other phytochemicals can be isolated and utilized in efficacious proportions in accordance with the present invention. These constituents can be combined with, or supplement, the raw sap to improve particular bioactivity of the present invention. The raw sap can be processed into a crystalline powder in an alternative embodiment, and the constituents can be utilized to engineer a particular consistent active constituent mix.

In some embodiments, fragrance, flavoring, carrier oils, preservatives are adjusted and mixed, or emulsified, with the product to provide improved taste, texture, transdermal bioavailability, and internal bioavailability.

In some embodiments, the present invention provides additives, including the constituents discussed above, can provide a longer shelf life without separation of oils and aqueous components.

Cannabidiol (CBD) Oil Oral Effects:

Cannabidiol (CBD) is one of many compounds, known as cannabinoids, in the cannabis plant. Researchers have been looking at the possible therapeutic uses of Cannabidiol (CBD).

Cannabidiol (CBD) oils are oils that contain concentrations of CBD. The concentrations and the uses of these oils vary. All cannabinoids, including CBD, produce effects in the body by attaching to certain receptors. The human body produces certain cannabinoids on its own. It also has two receptors for cannabinoids, called the CB1 receptors and CB2 receptors. CB1 receptors are present throughout the body, but many are in the brain.

The CB1 receptors in the brain deal with coordination and movement, pain, emotions, and mood, thinking, appetite, and memories, and other functions. THC attaches to these receptors. CB2 receptors are more common in the immune system. They affect inflammation and pain.

Researchers once believed that CBD attached to these CB2 receptors, but it now appears that CBD does not attach directly to either receptor. Instead, it seems to direct the body to use more of its own cannabinoids.

In one embodiment, the Cannabidiol (CBD) may benefit a person's health in a variety of ways such as natural pain relief and anti-inflammatory properties. People tend to use prescription or over-the-counter drugs to relieve stiffness and pain, including chronic pain.

Some people believe that CBD offers a more natural alternative. Authors of a study published in the Journal of Experimental Medicine found that CBD significantly reduced chronic inflammation and pain in some mice and rats. The researchers suggested that the non-psychoactive compounds in marijuana, such as CBD, could provide a new treatment for chronic pain.

Some promising evidence suggests that CBD use may help people to quit smoking. A pilot study published in Addictive Behaviors found that smokers who used inhalers containing CBD smoked fewer cigarettes than usual and had no further cravings for nicotine. A similar review, published in Neurotherapeutics found that CBD may be a promising treatment for people with opioid addiction disorders. The researchers noted that CBD reduced some symptoms associated with substance use disorders. These included anxiety, mood-related symptoms, pain, and insomnia. More research is necessary, but these findings suggest that CBD may help to prevent or reduce withdrawal symptoms.

Further, after researching the safety and effectiveness of CBD oil for treating epilepsy, the FDA approved the use of CBD (Epidiolex) as a therapy for two rare conditions characterized by epileptic seizures in 2018.

In the U.S., a doctor can prescribe Epidiolex to treat Lennox-Gastaut syndrome (LGS), a condition that appears between the ages of 3 and 5 years and involves different kinds of seizures, to treat Dravet syndrome (DS), a rare genetic condition that appears in the first year of life and involves frequent, fever-related seizures. The types of seizures that characterize LGS or DS are difficult to control with other types of medication. Therefore, the FDA specified that doctors could not prescribe Epidiolex for children younger than 2 years. A physician or pharmacist will determine the right dosage based on body weight.

Researchers are studying the effects of CBD on various neuropsychiatric disorders. Some studies noted that CBD has anti-seizure properties and a low risk of side effects for people with epilepsy. Therefore, some findings suggested that CBD may also treat many complications linked to epilepsy, such as neurodegeneration, neuronal injury, and psychiatric diseases.

Another study, published in Current Pharmaceutical Design, found that CBD may produce effects similar to those of certain antipsychotic drugs, and that the compound may provide a safe and effective treatment for people with schizophrenia. However, further research is necessary.

Some researchers have found that CBD may prove to combat cancer. Authors of a review published in the British Journal of Clinical Pharmacology found evidence that CBD significantly helped to prevent the spread of cancer. The researchers also noted that the compound tends to suppress the growth of cancer cells and promote their destruction. They pointed out that CBD has low levels of toxicity. They called for further research into its potential as an accompaniment to standard cancer treatments.

Further, doctors often advise people with chronic anxiety to avoid cannabis, as THC can trigger or amplify feelings of anxiousness and paranoia. However, authors of a review from Neurotherapeutics found that CBD may help to reduce anxiety in people with certain related disorders.

According to the review, CBD may reduce anxiety-related behaviors in people with conditions such as post-traumatic stress disorder, general anxiety disorder, panic disorder, social anxiety disorder, obsessive-compulsive disorder etc.

The authors noted that current treatments for these disorders can lead to additional symptoms and side effects, which can cause some people to stop taking them.

Many small-scale studies have looked into the safety of CBD in adults. They concluded that adults tend to tolerate a wide range of doses well.

Researchers have found no significant side effects on the central nervous system, the vital signs, or mood, even among people who used high dosages.

The most common side effect was tiredness. Also, some people reported diarrhea and changes in appetite or weight.

Therefore, the Cannabidiol (CBD) oil is obtained following all required testing, packaging, legal, and other requirements resulting in an organic highly bio available component to the final formulation.

Ajo Sacha is obtained in the form of a desiccated water soluble paste which is then dissolved in a sufficient quantity of hot water to add the aqueous solution to the carrier oil resulting in a final concentration of 600 mg paste per 60 ml (2 oz) finished product.

Sangre de Grado is added in the standardized aqueous commercial form at a rate of 0.5 ml per 60 ml bottle.

The ingredients are incorporated at the certified compounding laboratory using acceptable pharmaceutical methods with appropriate excipients such as emulsifiers, preservatives, fragrance, and flavorings, in any given carrier oil.

Standardized packaging and labeling procedures are used to create the final product.

The active ingredients, CBD oil and Ajo Sacha have been used topically to fight infectious lesions, shrink skin growths and chronic rheumatoid arthritis, repair nerve injuries, speed healing, and stop bleeding.

In some embodiments, packaging would be in a variety of containers of different sizes in glass ranging from 1 oz dropper bottles up to larger sizes depending on the market.

The composition described herein are useful for the treatment and prevention of a wide range of diseases, disorders or medical conditions, including, for example, including without limitation nausea, vomiting, cancer, muscle spasticity, pain, anorexia, AIDS, epilepsy, glaucoma, Crohn's disease, inflammatory bowel disease, multiple sclerosis, Amyotrophic Lateral Sclerosis (ALS), muscular dystrophy, bronchial asthma, post-traumatic stress disorder, mood disorders, cough and migraine headaches. Persons skilled in the art will recognize that the embodiments described herein may be used to treat many diseases, disorders or medical conditions that respond favourably thereto. Further, the composition is non-toxic in nature and used in medical, pharmaceutical and nutraceutical applications.
In embodiments, the composition comprises a pharmaceutical product; the dosage forms described herein provide clear separation from the confusion associated with traditional preparations of natural cannabinoid products. Preferably the concentration of 100-400 mg Ajo Sacha paste per 1 oz of water in the cannabinoid composition. Also preferably the concentration of aqueous Sangre de Grado in a concentration of 0.25 ml per 1 oz of the cannabinoid composition.
Further, in various embodiments, the CBD can be replaced by, or mixed with other cannabinoids in concentrations equal to the CBD concentrations expressed herein where the other cannabinoids include tetrahydrocannabinol, cannabigerol, and cannabinol. In other embodiments the CBD is in a whole plant extract of Cannabis sativa l (referred to herein as CBD oil). In yet another embodiment, isolated CBD is used to achieve the desired concentrations of CBD in the present cannabinoid composition. Likewise, various other cannabinoids can be isolated and added to supplement the present invention.

Any alterations and further modifications of the compositions and/or formulations described herein, which would normally occur to one skilled in the relevant art and having possession of this disclosure, are to be considered within the scope of the instant claims.

While particular elements, embodiments and applications of the present invention have been shown and described, it will be understood, of course, that the invention is not limited thereto since modifications can be made by those skilled in the art without departing from the scope of the present disclosure, particularly in light of the foregoing teachings.

1. A cannabinoid composition packaged in a dropper bottle for oral delivery, comprising:
water;
hemp extract containing Cannabidiol (CBD);
Ajo Sacha paste dissolved in the water;
Sangre de Grado dissolved in the water; and
an excipient selected from group consisting of emulsifiers, preservatives, fragrance, flavorings, carrier oil, and combinations thereof.

2. The cannabinoid composition of claim 1, wherein 100-400 mg of the Ajo Sacha paste dissolved in the water in a final concentration of 100-400 mg paste per 1 oz of cannabinoid composition.

3. The cannabinoid composition of claim 1, wherein 300 mg of the Ajo Sacha paste is dissolved in the water in a final concentration of 300 mg paste per 1 oz of cannabinoid composition.

4. The cannabinoid composition of claim 1, wherein the Sangre de Grado is added in the aqueous form at a concentration of 0.25 ml per 1 oz of the finished product.

5. The cannabinoid composition of claim 1, wherein the excipient is carrier oil selected from a group consisting of olive oil, avocado oil, coconut oil, and combinations thereof.

6. The cannabinoid composition of claim 1, wherein the Ajo Sacha paste contains detectable amounts of the organosulfur compounds consisting essentially of: diallyl disulphide, diallyl trisulphide, alliin, allicin, propylallyl, divinyl sulfide, diallyl sulfide, dimethyl sulfide, daucosterol, beta-sitosterol, fucosterol, stigmasterol, iridoides and isothiocyanates, naphthoquinones, alkaloids, and saponins.

7. The cannabinoid composition of claim 1, wherein the cannabinoid composition contains detectable amounts of the organosulfur compounds selected from the group consisting of: diallyl disulphide, diallyl trisulphide, alliin, allicin, propylallyl, divinyl sulfide, diallyl sulfide, dimethyl sulfide, daucosterol, beta-sitosterol, fucosterol, stigmasterol, iridoides and isothiocyanates, naphthoquinones, alkaloids, saponins, and combinations thereof.

8. The cannabinoid composition of claim 6, wherein the cannabinoid composition includes detectable amounts of ursolic acid.

9. A cannabinoid composition, comprising:
water;
hemp extract containing Cannabidiol (CBD);
100-400 mg Ajo Sacha paste per 1 oz of water;
the Ajo Sacha paste includes detectable amounts of at least one organosulfur compound selected from the group consisting of: diallyl disulphide, diallyl trisulphide, alliin, allicin, propylallyl, divinyl sulfide, diallyl sulfide, dimethyl sulfide, daucosterol, beta-sitosterol, fucosterol, stigmasterol, iridoides and isothiocyanates, naphthoquinones, alkaloids, saponins, and combinations thereof;
aqueous Sangre de Grado in a concentration of 0.25 ml per 1 oz of the of the cannabinoid composition; and
an excipient selected from a group consisting of emulsifiers, preservatives, fragrance, flavorings, carrier oil, and combinations thereof.

10. The cannabinoid composition of claim 9, wherein the cannabinoid composition is formulated into a topical cream, an oral spray, oral tincture, or dissolvable drops for time release delivery via oral mucosal membranes.

11. The cannabinoid composition of claim 9, wherein the cannabinoid composition is packaged in an oral tincture bottle with a dropper.

12. The cannabinoid composition of claim 9, wherein the cannabinoid composition includes detectable amounts of ursolic acid.

13. A cannabinoid composition packaged in a dropper bottle for oral delivery, consisting of:
water;
Cannabidiol (CBD);
Ajo Sacha paste to yield a concentration of 300 mg Ajo Sacha paste per 1 oz of cannabinoid composition;
the cannabinoid composition includes detectable amounts of the following organosulfur compounds: diallyl disulphide, diallyl trisulphide, alliin, allicin, propylallyl, divinyl sulfide, diallyl sulfide, dimethyl sulfide, daucosterol, beta-sitosterol, fucosterol, stigmasterol, iridoides and isothiocyanates, naphthoquinones, alkaloids, and saponins;
aqueous Sangre de Grado in a concentration of 0.25 ml per 1 oz of the finished product; and
an excipient selected from group consisting of emulsifiers, preservatives, fragrance, flavorings, carrier oil, and combinations thereof.

14. The cannabinoid composition of claim 13, wherein the cannabinoid composition is formulated into a topical cream, an oral spray, oral tincture, or dissolvable drops for time release delivery via oral mucosal membranes.

15. The cannabinoid composition of claim 13, wherein the cannabinoid composition is packaged in an oral tincture bottle with a dropper.

16. The cannabinoid composition of claim 13, the Ajo Sacha paste includes detectable amounts of Crofelemer derived from the latex of Croton lechleri.

17. The cannabinoid composition of claim 16, wherein the detectable amounts of Crofelemer are between 0.001 and 2.3% of the cannabinoid composition.
